(19) United States
(12) Patent Application Publication
(10) Pub. No.: US 2018/0184746 A1
(43) Pub. Date: Jul. 5, 2018

(54) HELMET CONSTRUCTION

(71) Applicant: Eric PLEBANI, Oakford, PA (US)

(72) Inventor: Eric PLEBANI, Oakford, PA (US)

(21) Appl. No.: 15/798,738

(22) Filed: Oct. 31, 2017

Related U.S. Application Data
(60) Provisional application No. 62/415,070, filed on Oct. 31, 2016.

(51) Int. Cl. A42B 3/22; A42B 3/04; A61F 9/06

(52) U.S. Cl. CPC A42B 3/225; A61F 9/065; A42B 3/042; A42B 3/0406

(57) ABSTRACT

A modular helmet may be configured and operative to allow simple interchangeability of both a function and an appearance of the helmet, without the need for tools. In some embodiments, a modular helmet generally comprises a base, headgear configured and operative to secure the base to a wearer's head, and a removable protection portion configured and selectively operative to attach to the base without tools; accordingly, a function of the helmet is selectively altered in accordance with a characteristic of the protective portion attached to the base.

### CROSS-REFERENCE TO RELATED APPLICATIONS

This application claims the benefit of the filing date of U.S. provisional patent application Ser. No. 62/415,070, filed Oct. 31, 2016, the disclosure of which is incorporated herein by reference in its entirety.

### BACKGROUND

### 1. Field

Aspects of the present disclosure relate generally to helmets that can be used in a variety of industrial applications.

### 2. Description of the Related Art

Prior art helmets are known which provide for different face screens and the like. However, many of these helmets require the use of tools to make adjustments and/or change components, face screens/shields, thereby making such changes cumbersome.

### SUMMARY

Aspects of the disclosed subject matter provide a helmet with multiple protection options that can be easily and quickly interchanged onto a helmet base without the use of external tools.

In accordance with one embodiment, a helmet includes a helmet base, headgear, and an external skin, and/or eye/face protection, which can consist of, but is not limited to, a clear tinted or filtered lens, various polycarbonate shields, and/or an auto darkening filter. The formerly listed protection options can be selected for use in a variety of industrial environments.

In accordance with another embodiment, the helmet base comprises a plastic injection molded shell and adjustable headgear that affixes to a user's head. The protective options can be easily and quickly interchanged to the helmet base without the use of tools. The helmet base with the desired protective option can act alone without an external skin to be used for welding and/or any environment where face and eye protection is necessary.

In accordance with another embodiment, an externally thermoformed skin can be attached to the helmet base to give the appearance and color of a character, cartoon, figure, animal, robot, licensed character, cobranded theme, etc. This external skin will give the perception of a character or theme through the use of a three dimensional shape with applied color or graphic. The external skin can, similarly to the protective options, be easily interchanged with other characters or themes, without the use of tools to provide the helmet with various aesthetic options and variations.

In accordance with some aspects of the disclosed subject matter, a welding helmet may be configured to allow modular interchangeability of both the function and appearance of the helmet.

Aspects of the disclosed helmet include: the ability to interchange components of the helmet, which will alter the inherent function of the helmet, for example: exchanging the inner base shell will allow the helmet to act as a welding helmet or general face protection; the ability to interchange external skins, which has a variety of three dimensional shapes, color, and styles for providing a variety of selections, so that the wearer of the helmet is able to selectively and quickly replace the external skin with a different skin for the purpose of altering the aesthetic appearance; and utilization of integrated hardware components for the purpose of quick and simple attaching, detaching or interchanging of components to allow modularity of the helmet

Other objects and features of the disclosed embodiments will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

The disclosed subject matter and some relevant technical environments are explained below with reference to the drawing figures. It is to be noted that the figures show particular variants, but the present disclosure is not, however, restricted to the embodiments illustrated. The same components in the figures are given the same reference signs. In the drawings:

FIG. 1 is a set of views of helmet components and assemblies;

FIG. 2 is a view of a helmet base without any protective mask attached;

FIG. 3 is a rear view of a helmet assembly;

FIGS. 4A and 4B are perspective views of a helmet assembly;

FIG. 5 is an exploded view of mounting hardware for attaching the helmet base to a protective face mask to form the helmet assembly;

FIG. 6 is a rear view of a helmet assembly;

FIG. 7 is an exploded view showing components of a helmet assembly;

FIG. 8 is a rear view of a face shield insert with ADF filter;

FIG. 9 is a view showing a helmet assembly and two types of protective face masks;

FIG. 10 is a view showing connecting holes used to accommodate mounting hardware; and

FIG. 11 is a view showing a skin type mask assembly.

### DETAILED DESCRIPTION

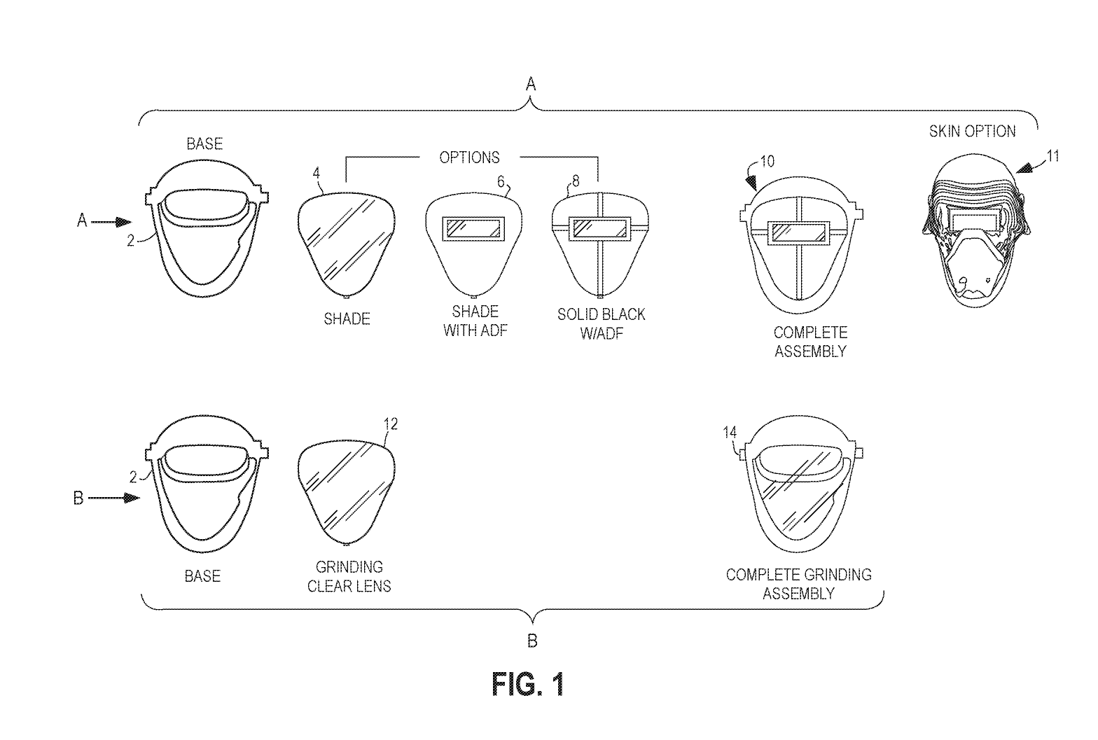

As shown in FIGS. 1-11, a modular helmet is provided for a wide variety of environments.

FIG. 1 shows different configurations available for adding optional components to a helmet base 2. As shown, the helmet base 2 can be combined with different protective shields, which may have a lens in an opening in the area of the user's eyes, or may in its entirety form a lens, to be used in a variety of industrial environments. Additionally, numerous external skins can be combined with the helmet base 2 to give the helmet a unique, distinct and changeable appearance.

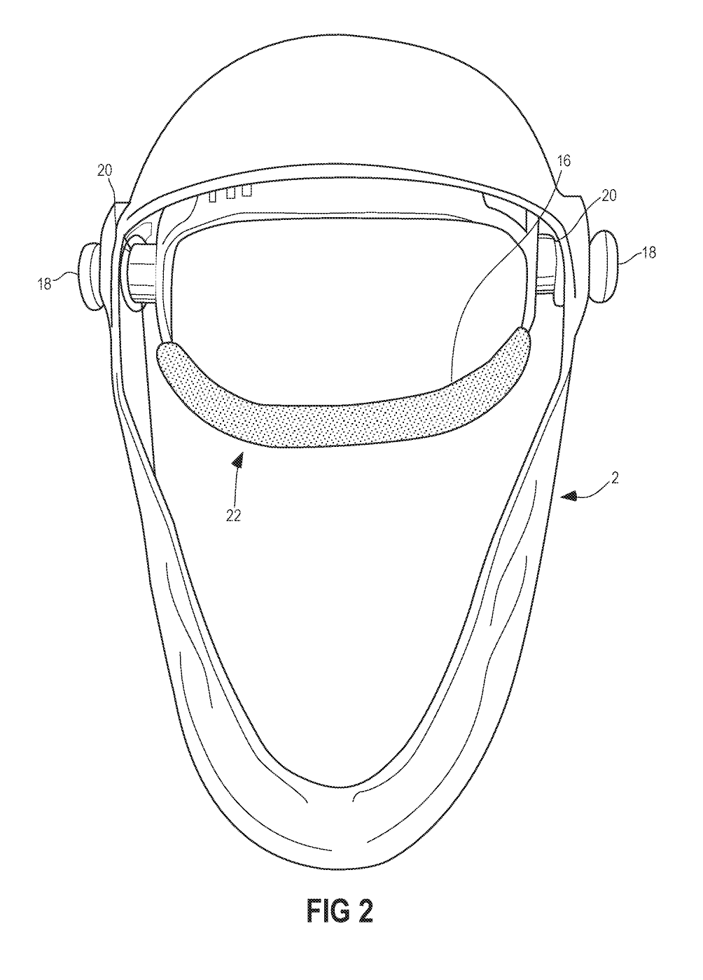

The helmet base 2 is shown in FIGS. 1 and 2. The helmet base 2 has an opening where the protective options attach via two clips, one on each side of the helmet base 2. These clips, discussed in more detail below, go through the helmet base 2 and the protective lens to firmly attach and align the two units together.

As shown in FIG. 1, Row A, exemplary protective shields include a shade 4, which is a solid translucent shield that has a high shading capability to protect the user's eyes from bright light. The shade 6 having an auto dimming feature (ADF) is also shown in FIG. 1. The auto dimming feature will be discussed further below. The shade 8 is the same as shade 6 but is colored black.

The completed helmet assembly 10 is shown in FIG. 1 with the protective shield attached to the helmet base 2. At the end of Row A of FIG. 1 is shown a helmet assembly 11 with a skin option, in which the attached shield comprising a skin of, for example, a decorative type, is attached to the helmet base 2.

Row B of FIG. 1 shows the helmet base 2 and a clear grinding lens 12. In the case of grinding, for example, shading may not be necessary, in contrast to welding, in which shading or filtering would generally be required. The complete grinding assembly 14 using the fully clear grinding lens 12 is shown in Row B of FIG. 1.

FIG. 2 shows the helmet base 2, without any shade or face mask attached. The helmet base includes the head band 16 and head band adjustment outer knobs 18, one on each side of the helmet base 2. The outer knobs 18 allow for a rotational loosening or tightening of the head band tilt adjusters 20. The tilt adjusters 20 allow for stepped adjustment of the helmet on the user's face. That is, by the use of the tilt adjusters, and the detents formed thereon, rotation of the helmet base 1 can be adjusted, using the detents, to, for example three positions: a completely closed position, in which the face mask of the helmet is completely in the lowered position to provide protection, for example during welding or grinding, a partially up position, which allows the user a partial lower view, and a fully up position, when the protective shield is not needed.

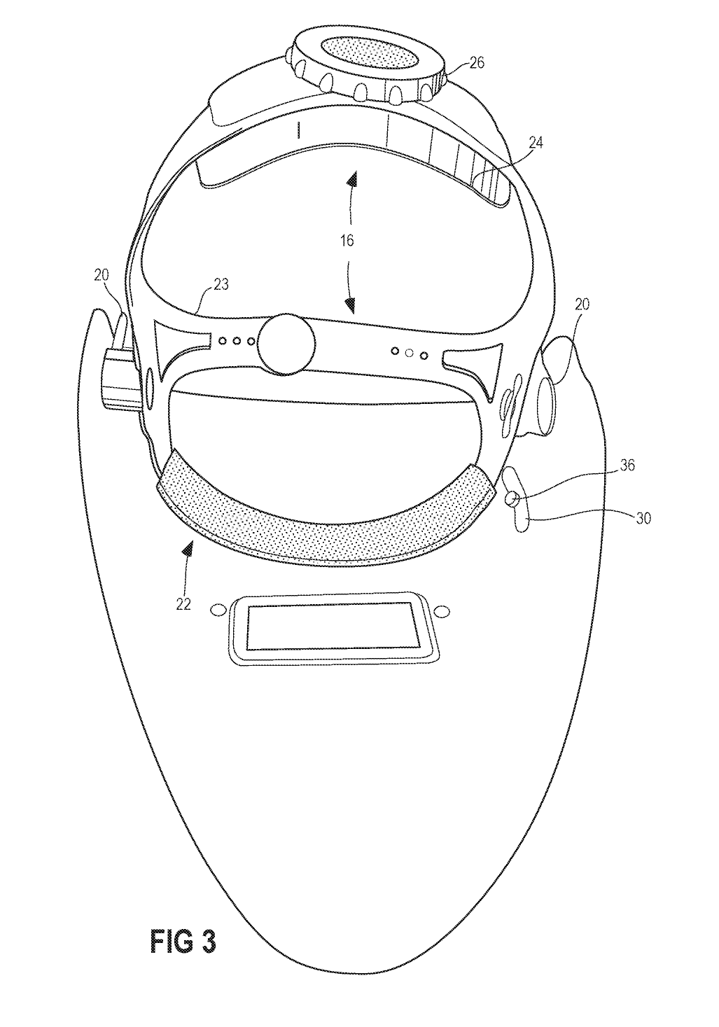

As illustrated in FIG. 3, the head band includes a forehead support portion 22, which rests against the user's forehead when the user wears the helmet, a head top support portion 23, which rests on the top of the user's head during use, and an adjustable head back support portion 24, which rests on the back of the user's head. The head back support portion 24 has an adjustment knob 26, the rotation of which causes the head back support portion 24 to adjust to accommodate different head sizes.

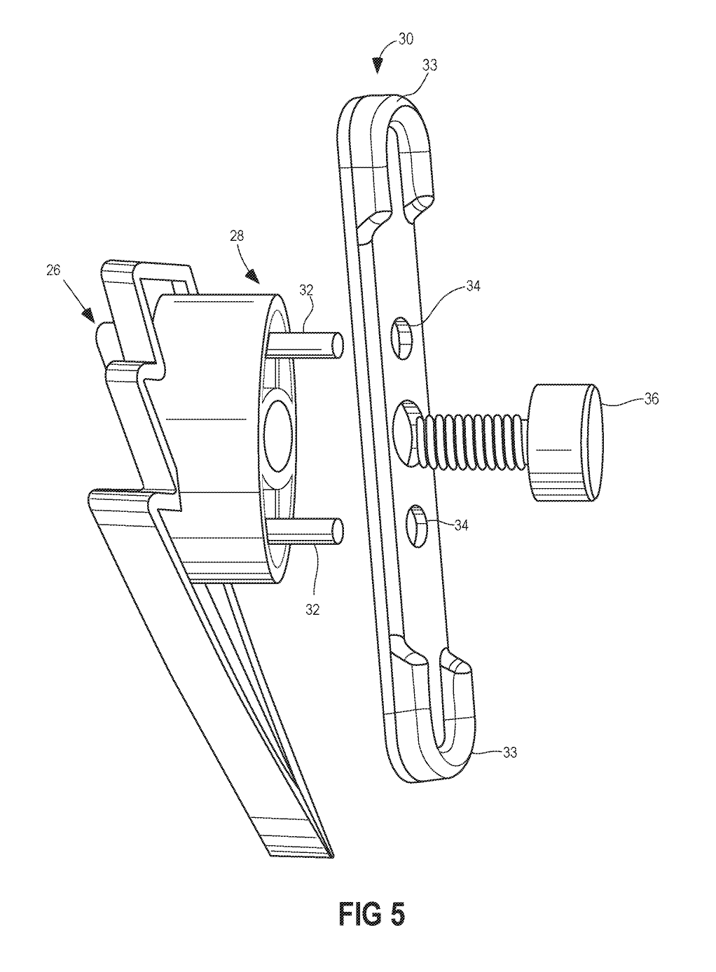

Attachment of the protective shields to the helmet base 2 is effected using two instances of mounting hardware 25, one instance on each side of the helmet proximate a user's ears. Each instance of mounting hardware 25 includes a locating clip 26, in the figures shown as having a lightning bolt type appearance, a locating washer 30 and a thumb screw 36. One instance of the mounting hardware 25 is illustrated in FIG. 5.

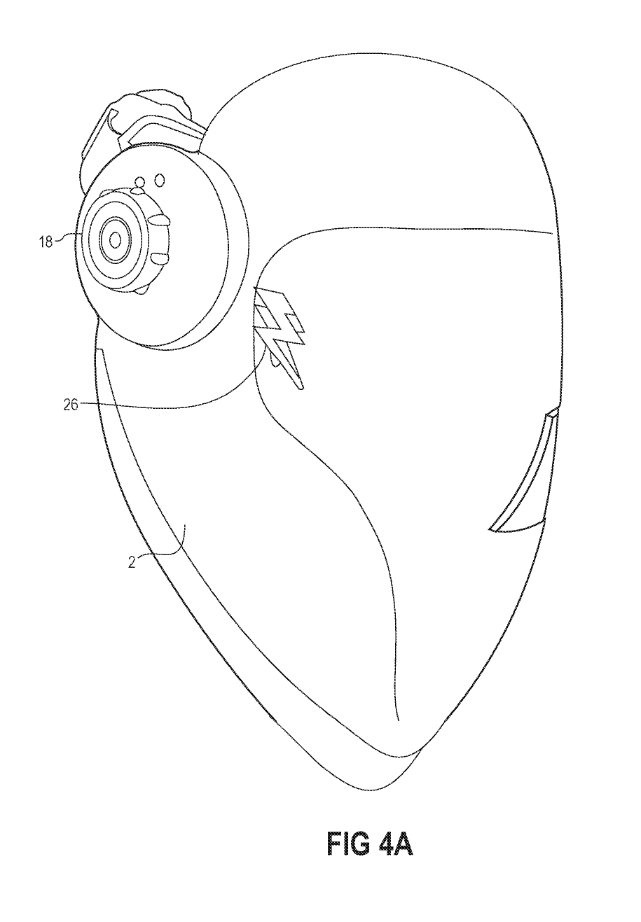

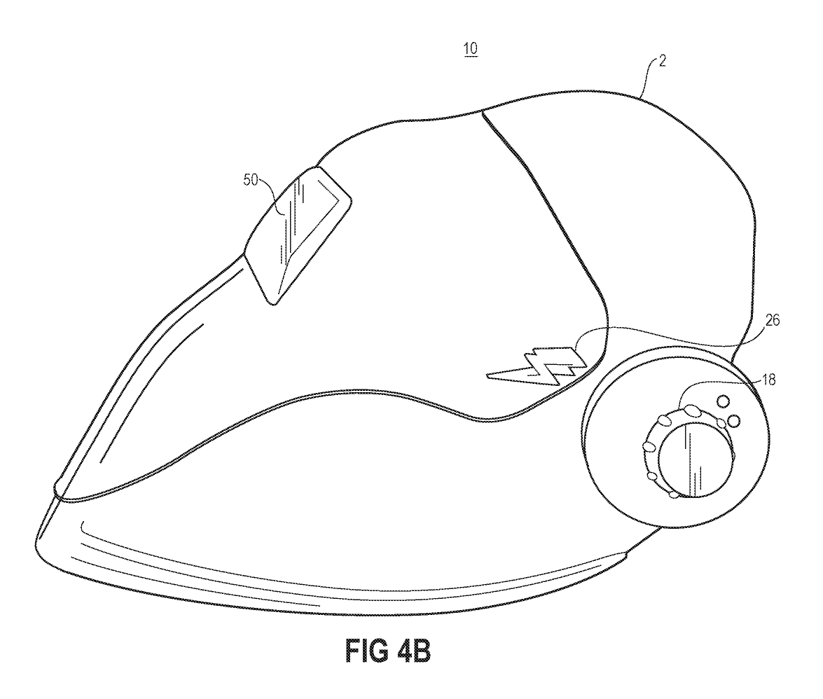

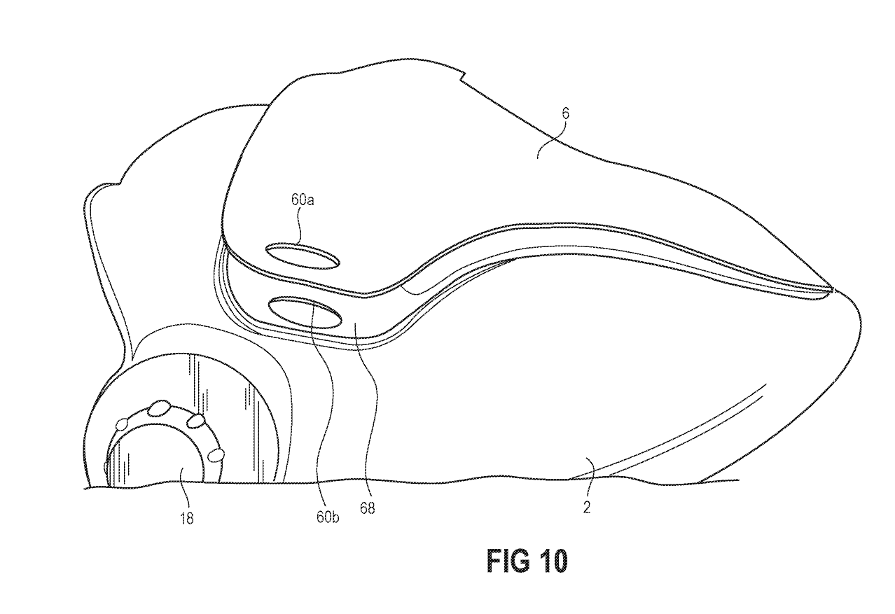

One of the locating clips 26 can be seen, for example, in FIGS. 4A and 4B. FIG. 5 also shows one of the locating clips 26, which, in use, is arranged on the outside of a protective shield to be attached to the helmet base 2. The locating clip 26 includes an integrally formed connecting projection 28. The reversible tensioning locating washer 30 is arranged on the inside of the helmet base 2, at a flange of the helmet base 2 on which an edge of the protective shield rests. The flange provides an area on which a portion of the protective shield can be supported, as shown in FIG. 10, discussed below.

The locating clip 26 has a connecting projecting portion 28 having projections 32. The locating washer 30 has recesses 34 which are positioned to mate with respective projections 32. Both the protective shield and the helmet base 2 have corresponding holes to allow the projections 32 and recesses 34 to mate with one another.

To provide a secure, yet removable, affixing of the protective shield to the helmet base, a thumb screw 36 is provided. As can be seen from FIGS. 3 and 5, the thumb screw 36 is located on the inner surface of the helmet base 2 and, when tightened, securely affixes the protective shield to the helmet base 2 through a hole formed through both the flange of the helmet base 2 and the protective shield. Advantageously, screw 36 requires no tools to tighten or loosen.

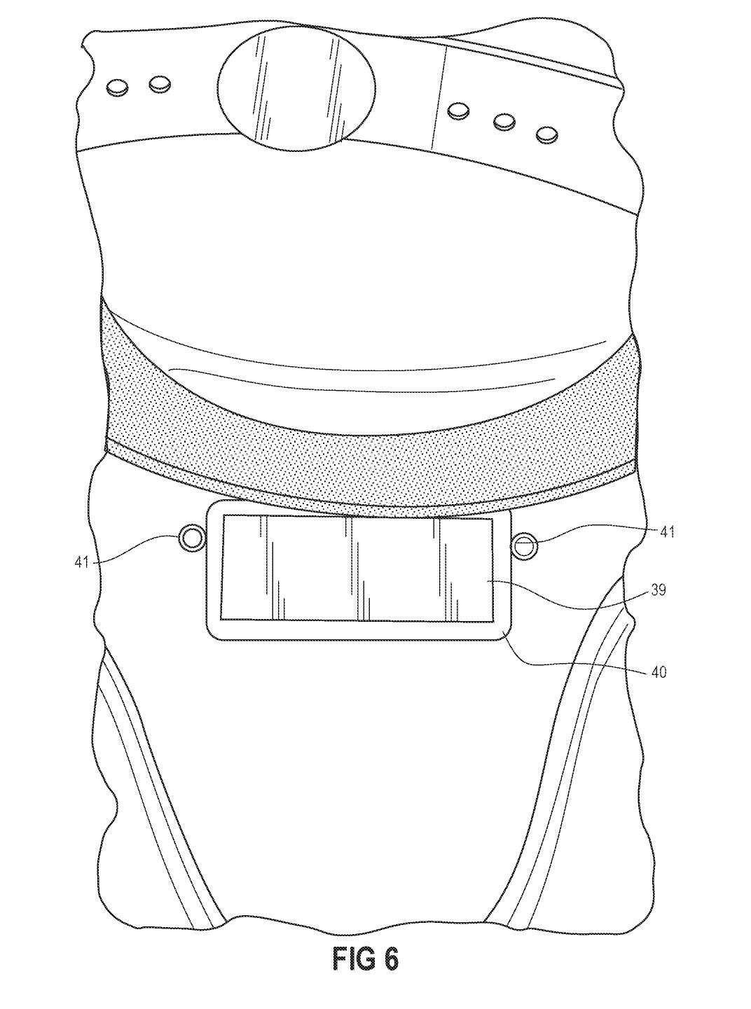

FIG. 6 is a view from the back, i.e., inside, of the helmet with an ADF shade 6 attached to the helmet base 2. The ADF shade 6 includes an auto dimming filter 39 that is configured to dim in the presence of extremes of light. The auto dimming filter 39 is affixed to the ADF shade 6 via an auto dimming filter bracket 40. The bracket is affixed to the ADF shade 6 by two ADF bracket thumb screws 41, which preferably can be tightened or loosened without the need for tools.

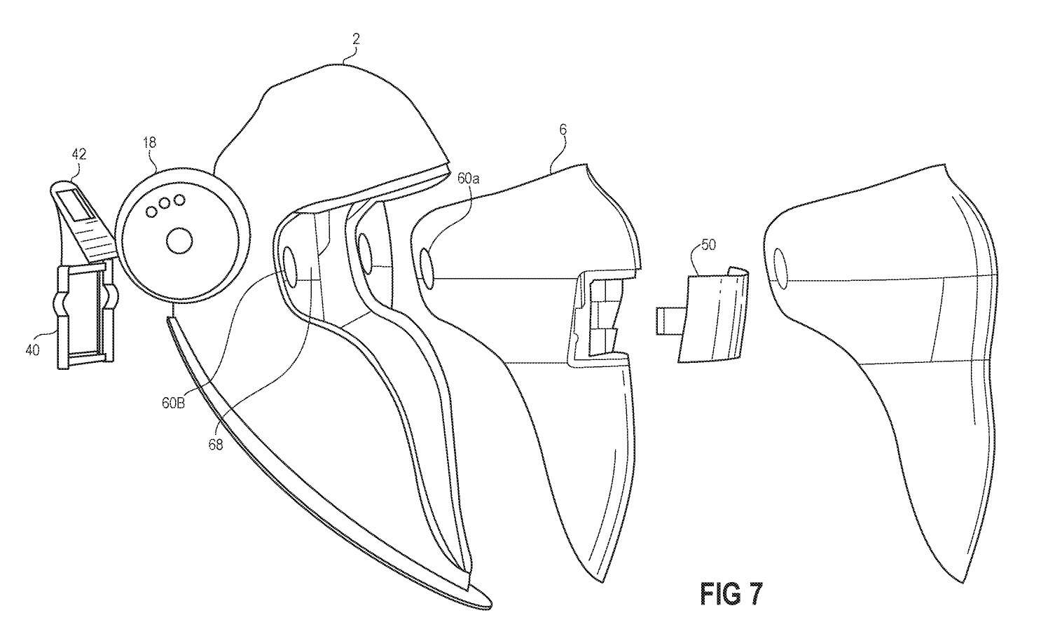

FIG. 7 is an exploded view of the helmet, showing the ADF filter bracket 40. The top portion 42 is an optional portion of the bracket used in the case of the ADF with an adjustment feature. The top portion 42, as will be discussed below, houses the control knobs for adjustment. Also shown in FIG. 7 are the helmet base 2, and an ADF shade 6 having a lens protector 50.

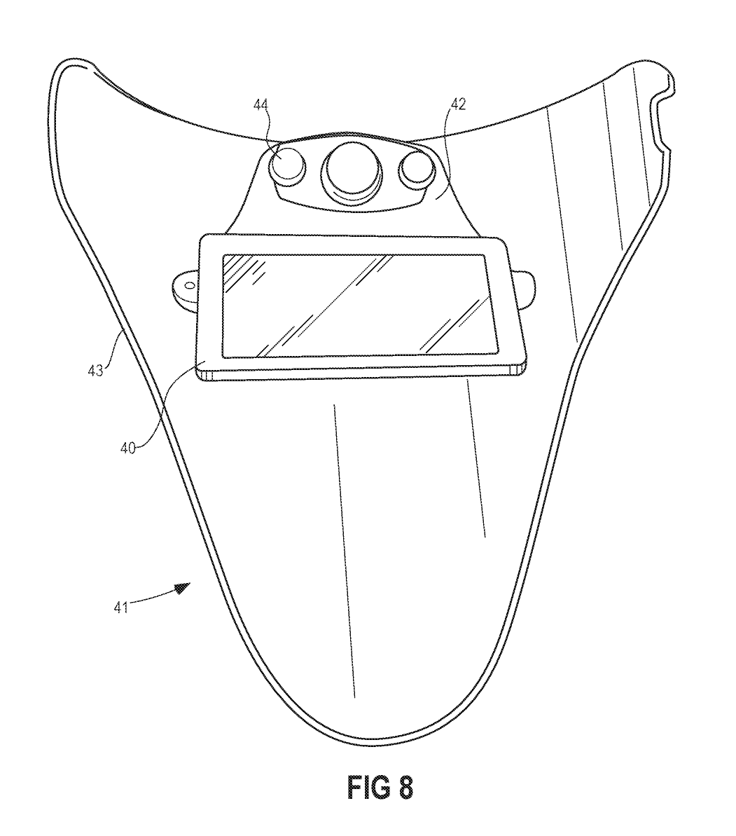

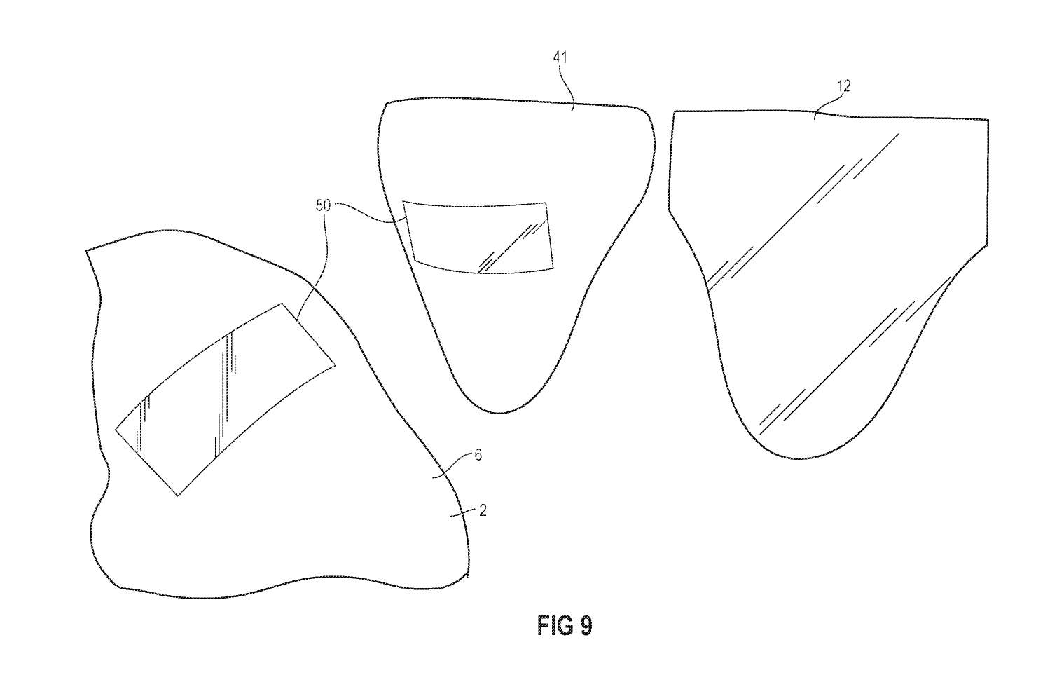

FIG. 8 is a rear view of an adjustable ADF shade 41, which is also shown in FIG. 9. As can be seen in FIG. 8, the knobs 44 are located in the upper portion 42 of the bracket 40. As discussed above, the upper portion 42 of the ADF bracket only occurs when the bracket is for the adjustable ADF shade 41. As can be seen in FIG. 8, the upper portion 42 of the bracket 40 houses the adjustment knobs which allow the dimming feature to be adjustable.

FIG. 9 shows a base helmet assembly with an ADF shade 6 installed onto the helmet base 2, and also shows examples of an unattached adjustable ADF shade 41 and an unattached grinding shield 12. Both the adjustable ADF shade 41 and the ADF shade 6 have a lens protector 50 at an outer surface thereof, to protect the lens in the eye area.

FIG. 10 is a view of an ADF shade 6 and a helmet base 2 showing the holes 60a and 60b through which the mounting hardware 25 is inserted in attaching the helmet base 2 to an ADF shade 6. It should be noted that the same holes are found in the other shades and face shields discussed above, as well as in the skin 11.

Making reference also to the description related to FIGS. 3-5 and 7, as can be seen in FIG. 10, flanges 68 are formed on the helmet base 2. The flanges 68 are portions of the helmet base 2 that are slightly lower than the surface of the nearby area of the helmet base 2. The flanges 68 function as a platform on which upper outer portions of the shades or lenses can be supported when the assembly 10 is in the assembled state.

As discussed above, connection is effected using two instances of mounting hardware 25, one instance on each side of the helmet. To connect the components of the assembly 10, the integrally formed connecting projection 28 is pushed, from the outside, through the hole 60a formed in the upper outer portion of the shade or lens. The reversible tensioning locating washer 30, is, in use, is arranged on the inside of the helmet base 2, on the inner side of the flange 68 at the location of the hole 60b of the helmet base 2 on which flange 68 the edge of the protective shield (shade or lens) rests. As discussed above, the flange 68 provides an area on which a portion of the protective shield (shade or lens) can be supported.

The locating clips 26 connecting projecting portion 28 has projections 32, which are placed through the hole 60a. The locating washer 30, as can be seen in FIGS. 3 and 5, has recesses 34, which are positioned to mate with respective projections 32. The locating washer 30 is positioned on the inner side of the helmet base 2 at the hole 60a formed in the flange 68. The two holes 60a and 60b are lined up and the connecting projecting portion 28 is pushed through the lined up holes to allow the projections 32 and the recesses 34 to mate with one another.

The reversible tensioning locating washer 30 has the unique ability to give two different lengths of depth, allowing the hardware to be used for all configurations. This feature is a product of upstanding walls or spacers 33 that are added to one side of the washer. In order to increase the depth of the spacing provided by the washer 30, the user simply reverses the washer 30 and reattaches the hardware.

To provide a secure, yet removable, affixing of the protective shield to the helmet base using the mounting hardware 25, the thumb screw 36, is located on the inner surface of the helmet base 2. When tightened, the thumb screw 36 securely affixes the protective shield (shade or lens) to the helmet base 2 through the lined up holes 60a and 60b formed through protective shield (shade or lens) and the flange 68 of the helmet base 2, respectively. Advantageously, screw 36 requires no tools to tighten or loosen.

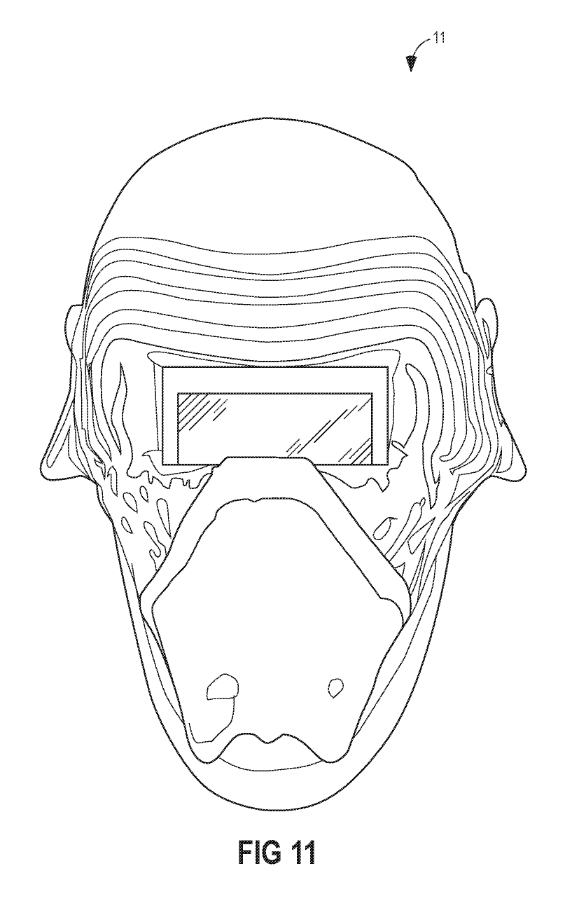

Instead of, or in addition to, the protective shield, a decorative skin can be affixed to the helmet base 2 to provide a helmet assembly 11 with a skin option, in which the attached shield comprising a skin of, for example, a decorative type, is attached to the helmet base 2. An example of such a skin option helmet assembly can be seen in FIG. 11. Similar to the other embodiments, the skin can be attached to the helmet base 2 using the same mounting hardware 25.

The skin, for example a character or themed shaped skin, can be attached to the helmet base 2 with or without the protective shield to give the appearance and color of a character, cartoon, figure, animal, robot, licensed character, cobranded theme etc. This external skin will give the perception of a character or theme through the use of a three dimensional shape with applied color or graphic. The external themed skins can, similarly to the protective options, be easily interchanged with other characters or themes, with the use of no tools, in order to give the helmet a new and distinct appearance.

The external skins are preferably made of a thermoformed plastic or other material with an identifying shape and color that will attach to the base helmet. The same clips used to attach the protective options will preferably be used to attach the external skin. The external skins will utilize three dimensional shape and color/graphic to demonstrate the character or theme. The external skins can easily be removed and installed without the use of tools.

Other possible upgrades for the helmet assembly described above can include a cooling band for head gear that utilizes cooling technology, e.g., from the fitness industry. In such a variation, the forehead support portion 22 can comprise a cooling gel, for example, or any other appropriate cooling material.

Other options are to attach, for example to the side mounted clips LED Lighting and/or for example a GoPro camera mount. Other variants can include the inclusion in the helmet assembly a Bluetooth speaker and microphone and/or a small screen for viewing incoming calls, surface temperatures, etc.

The auto-darkening lens discussed above is responsible for protecting the user's eyes from the dangerous high intensity light that is a by-product of welding. At least two types of such lenses may be used, namely, one that has a controller to give the user additional filtering control and one that does not. The controlled version may have four external adjustments, three located on the control unit (sensitivity, delay, and shade) a one control on the filter itself (shade switch).

The other available lens can be a standard non-adjustable Auto-Darkening filter. Both lens options will utilize the same mounting hardware regardless of which lens is selected, further employing the modularity of the product. The lens will be held into place between the inner shell and a holder using two thumb screws. The inner shell will have two inserts that the thumb screws will thread into. This method of attachment will be used for all of the configurations of lenses and inner shells.

The external skins will be designed to give the perception of a character, animal, theme, etc. via the use of three dimensional shape and color. In addition, the external skin may offer a unique function or purpose for use in specific environments. The external skin will attach to the base helmet by utilizing the same hardware that is used to assemble the base helmet. Therefore, the external skins can be attached, detached or interchanged onto the base helmet using no tools or additional hardware. The interchangeability of the external skins will allow the user to quickly change the three dimensional appearance of the product.

Several features and aspects of the disclosed subject matter have been illustrated and described in detail with reference to particular embodiments by way of example only, and not by way of limitation. Those of skill in the art will appreciate that alternative implementations and various modifications to the disclosed embodiments are within the scope and contemplation of the present disclosure. Therefore, it is intended that the invention be considered as limited only by the scope of the appended claims.

What is claimed is:

1. A modular helmet comprising:
a base,
headgear configured and operative to secure the base to a wearer's head, and
a removable protection portion configured and selectively operative to attach to the base without tools;
wherein the protection portion is positioned to protect the wearer's face when the headgear is secured to the wearer's head and whereby a function of the helmet is selectively altered in accordance with a characteristic of the protective portion.

2. The modular helmet of claim 1 wherein the characteristic of the protective portion is determined by one of a lens, a polycarbonate shield, and an auto darkening filter.
